(19) United States
(12) Patent Application Publication
(10) Pub. No.: US 2008/0220403 A1
(43) Pub. Date: Sep. 11, 2008

(54) SYSTEM AND METHOD FOR MANAGING DIABETES

(75) Inventors: Cynthia Robin Marling, Athens, OH (US); Frank Lee Schwartz, Vienna, WV (US)

Correspondence Address: CALFEE HALTER & GRISWOLD, LLP, 800 SUPERIOR AVENUE, SUITE 1400, CLEVELAND, OH, 44114, US

(73) Assignee: OHIO UNIVERSITY, Athens, OH (US)

(21) Appl. No.: 12/032,021

(22) Filed: Feb. 15, 2008

Related U.S. Application Data
(60) Provisional application No. 60/901,730, filed on Feb. 16, 2007.

(51) Int. Cl. G09B 23/28; A61B 19/00; A61K 9/22

(52) U.S. Cl. 434/262; 128/898; 604/890.1

(57) ABSTRACT

A system and method are provided for automatically analyzing data for a patient with type 1 diabetes on insulin pump therapy and recommending appropriate therapeutic adjustments to the patient. In the system and the method, case-based reasoning is used as a primary reasoning modality in determining the therapeutic adjustments.

### RELATED APPLICATION

The present application is being filed as a non-provisional patent application claiming priority under 35 U.S.C. § 119(e) from, and any other benefit of, U.S. Provisional Patent Application No. 60/901,730 filed on Feb. 16, 2007, the entire disclosure of which is herein incorporated by reference.

### FIELD

The invention relates generally to disease management and, more particularly, to a system and method for managing diseases in patients.

### BACKGROUND

Diabetes mellitus (hereinafter “diabetes”) is a metabolic disorder/disease that affects carbohydrate metabolism. Diabetes is characterized by persistent high blood glucose levels (i.e., hyperglycemia). Diabetes requires medical diagnosis, treatment and lifestyle changes. The World Health Organization recognizes three main forms of diabetes: type 1, type 2 and gestational diabetes (or type 3) which occurs during pregnancy. Type 1 diabetes is generally due to autoimmune destruction of insulin-producing cells, while type 2 diabetes and gestational diabetes are due to insulin resistance by tissues.

Diabetes is a treatable but chronic condition. Diabetes is characterized by long-term complications such as cardiovascular disease, chronic renal failure, retinal damage, nerve damage and gangrene. Often, managing diabetes involves insulin replacement. Insulin is a hormone secreted by the pancreas which regulates the uptake of glucose into most cells (primarily muscle and fat cells) from the blood. If the amount of insulin available is insufficient, if cells respond poorly to the effects of insulin or if the insulin itself is defective, glucose will not be handled properly by body cells or stored appropriately in the liver and muscles. As a result, a patient suffering from diabetes can have persistent high levels of blood glucose (hyperglycemia), poor protein synthesis and other metabolic problems (e.g., acidosis).

Because patients with type 1 diabetes cannot produce their own insulin, they depend on exogenous insulin to survive. Type 1 diabetes also requires careful monitoring of blood glucose levels using blood testing monitors. Lifestyle adjustments (e.g., diet and exercise) can also be part of the treatment for controlling type 1 diabetes. Replacement insulin can be injected into the body using a syringe of via an insulin pump, which allows infusion of basal insulin 24 hours a day at preset levels, with the ability to program a push dose (i.e., a bolus) of insulin as needed (e.g., at meal times). Basal insulin is intended to replace the insulin that is released continuously by the pancreas throughout the day during the fasting state in a non-diabetic individual. Bolus insulin is intended to replace the insulin that is released periodically by the pancreas in response to rising glucose levels following the ingestion of food by a non-diabetic individual.

Currently, the treatment for type 1 diabetes must be continued indefinitely. Elevated blood glucose levels or hyperglycemia, resulting from too little insulin, can lead to numerous complications over time including blindness, neuropathy and heart failure. Low levels of blood glucose or hypoglycemia, resulting from too much insulin, can cause a patient to experience weakness, confusion, dizziness, sweating, shaking and, if not treated promptly, can lead to seizures or episodes of unconsciousness. Thus, patients with type 1 diabetes must keep their blood glucose levels as close to normal as possible, avoiding both hyperglycemia and hypoglycemia, to maintain their health and avoid serious complications. These patients must continuously monitor their blood glucose levels and daily activities in order to maintain good glycemic control. Traditionally, the patients maintained this information in daily logs, which they presented to a physician three or four times a year at office visits for analysis and review.

Currently, patients with type 1 diabetes that are on insulin pump therapy have access to continuous glucose monitors that record glucose values periodically (e.g., every five minutes). Software allows these patients to track their blood glucose levels and send this data to the physician every few days. Current data gathering software does not allow adequate documentation of life events that contribute to specific glucose levels. Furthermore, current data gathering software does not recognize repetitive patterns of glucose excursions. Further still, current data gathering software lacks the ability for automatic pattern analysis of the large volumes of glucose data generated by the continuous glucose monitors. Yet further still, current data gathering software retains no systematic record of previous causes or solutions for a particular problem or for each patient. Accordingly, the physician must manually analyze a large amount of data for each patient, usually with incomplete patient information, and on a more frequent basis than before, which places a tremendous burden on the physician.

Consequently, there is a need in the art for a system that will automatically analyze a patient's data and recommend a therapeutic adjustment when necessary based on the data. The recommended adjustment should be approximately the same as an accurate adjustment that a physician manually reviewing the data would make.

### SUMMARY

In view of the above, it is an exemplary aspect to provide a system and a method for automatically analyzing data for a patient with diabetes (e.g., a patient with type 1 diabetes on insulin pump therapy or a patient with type 2 diabetes on oral medications) and recommending appropriate therapeutic adjustments for the patient. The therapeutic adjustments can be communicated to a patient, to an intermediary (e.g., physician, caregiver) and/or to a device (e.g., an insulin pump) associated with the patient. In the system and the method, case-based reasoning (hereinafter “CBR”) is used as a primary reasoning modality in determining the therapeutic adjustments.

It is another exemplary aspect to provide a system and a method for compiling and maintaining a case library for supporting a CBR approach to determining therapeutic adjustments to recommend to a patient with diabetes.

It is an exemplary aspect to provide a system and a method for determining how similar a current problem experienced by a patient with diabetes is to a previous problem experienced by the same patient or a different patient.

It is yet another exemplary aspect to provide a system and a method for determining how similar a patient with diabetes is to another patient with diabetes.

### BRIEF DESCRIPTION OF THE DRAWINGS

The above aspects and additional aspects, features and advantages will become readily apparent by describing in detail exemplary embodiments thereof with reference to the attached drawings, wherein like reference numerals denote like elements, and:

FIG. 1 is a diagram of a system for automatically determining an appropriate insulin dosage adjustment or other treatment recommendation, according to an exemplary embodiment.

FIG. 2 is a screenshot from a program outputting a graph that plots glucose readings over a period of time, according to an exemplary embodiment.

FIG. 3 is a screenshot from a program displaying a web-based user interface for inputting data on a patient, according to an exemplary embodiment.

FIG. 4 is a flowchart illustrating a method of forming a library of cases, according to an exemplary embodiment.

FIG. 5 is screenshot from a program outputting a graph that that plots various types of data on a patient over a period of time.

FIG. 6 is a diagram illustrating an exemplary case, according to an exemplary embodiment.

FIG. 7 is a diagram illustrating an exemplary library, according to an exemplary embodiment.

### DETAILED DESCRIPTION

While the general inventive concept is susceptible of embodiment in many different forms, there are shown in the drawings and will be described herein in detail specific embodiments thereof with the understanding that the present disclosure is to be considered as an exemplification of the principles of the general inventive concept. Accordingly, the general inventive concept is not intended to be limited to the specific embodiments illustrated herein.

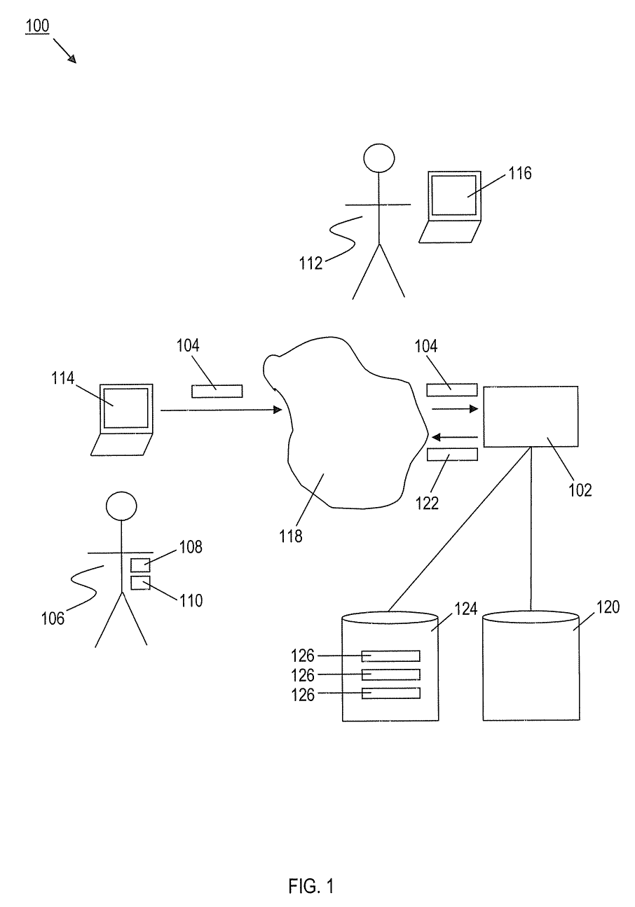

According to an exemplary embodiment, a system 100 for automatically analyzing data for a patient with diabetes, such as a patient with type 1 diabetes on insulin pump therapy, and recommending appropriate therapeutic adjustments is disclosed. As shown in FIG. 1, the system 100 includes a server 102 that receives patient data 104 from a patient 106 with type 1 diabetes on insulin pump therapy. A pump 108 delivers basal insulin at varying rates throughout the day to the patient 106, while allowing the patient 106 to instruct the pump 108 to deliver additional doses of insulin (i.e., boluses) as needed (e.g., before meals). Typically, the patient 106 on insulin pump therapy tries to maintain blood glucose levels between assigned high and low targets and monitors actual blood glucose levels through finger stick testing from four to six times a day. The amount of bolus insulin depends on many factors including, for example, the amount of carbohydrates being consumed, the current blood glucose level, the anticipated level of physical activity and the historical response of the patient 106 to a particular dose of insulin. The waveform of the bolus can also vary (e.g., sine, square, dual-wave) depending, for example, on the type of meal. These factors are not the same as those for type 1 diabetics on traditional (i.e., non-pump) intensive insulin therapy, who use syringes to inject themselves (e.g., three or four times a day). With traditional insulin therapy, there is less opportunity to fine tune control or to account for variations in the daily routine of the patient 106.

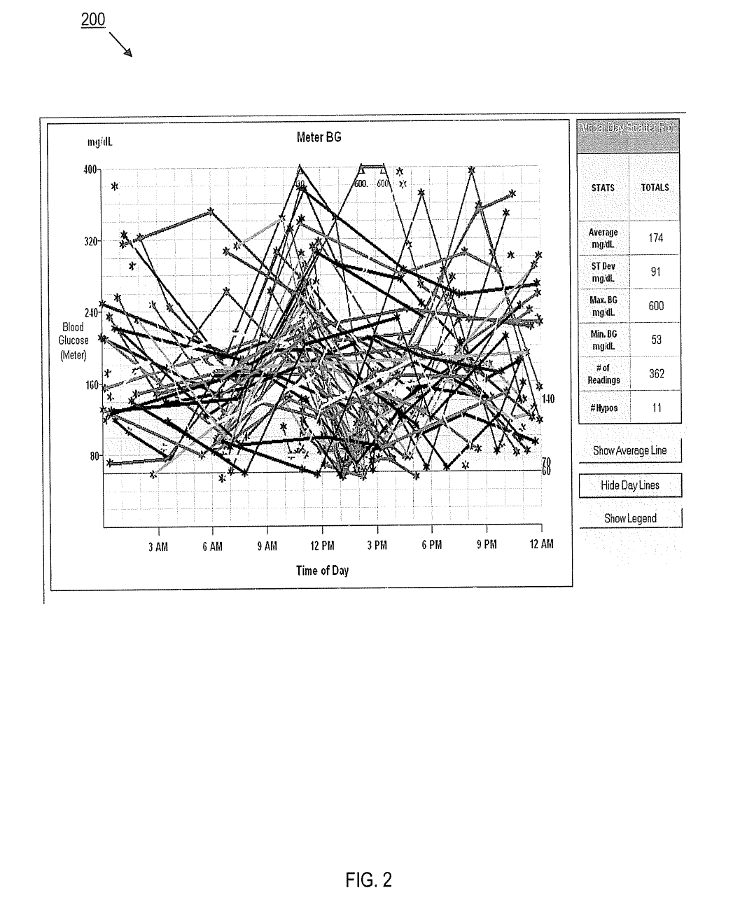

The patient 106 can also use a glucose meter 110 to monitor his or her blood glucose levels. The glucose meter 110 records the glucose level of the patient 106 periodically (e.g., every five minutes). Accordingly, the glucose meter 110 expands upon the number of blood glucose level readings available from routine daily finger sticks, which typically average six per day for a patient on insulin pump therapy. Software running on or interfacing with the pump 108 and/or the glucose meter 110 facilitates collection and transmission of the blood glucose level readings, as well as other data determined from monitoring the patient 106. As shown in FIG. 2, this monitored data (displayed as a graph 200) can present an overwhelming amount of data making accurate manual analysis difficult if not impossible.

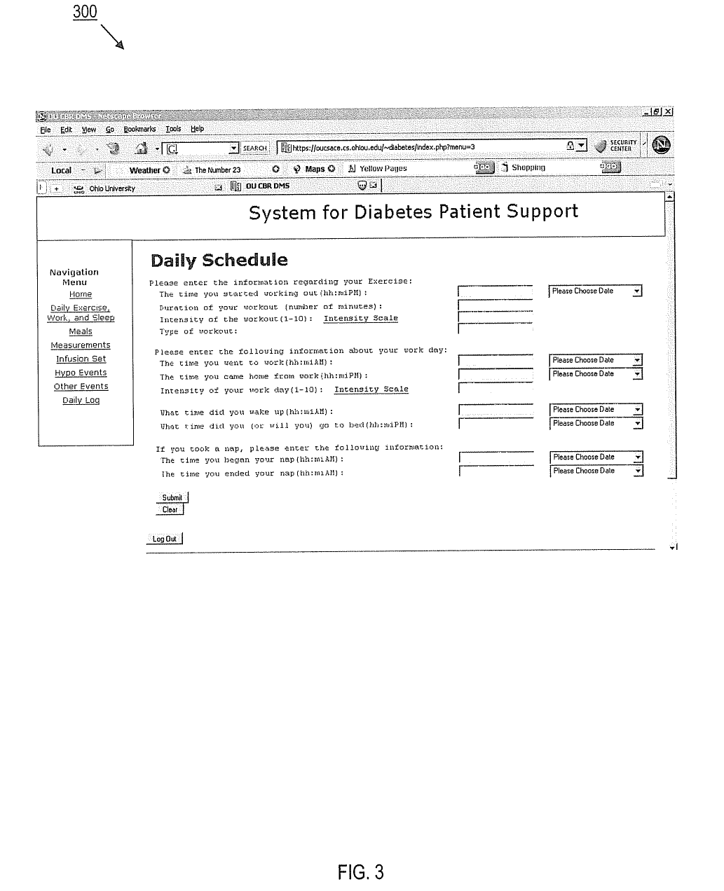

The patient data 104 includes the monitored data (e.g., the blood glucose levels) and personal data for identifying the patient 106. The patient data 104 also includes information for determining a therapeutic adjustment, if necessary, in the insulin dosage of the patient 106. By way of example, this information includes occupational information, information on the pump 108, insulin sensitivity, carbohydrate ratios, HbAlc as a measure of long-term blood glucose control, complications from diabetes, other chronic diseases, medications, family history of diabetes and typical daily schedules for work, exercise, meals and sleep. The information can be provided by the patient 106 and/or a physician 112 of the patient 106. If the information is provided by the physician 112, it can be obtained from the patient's medical records or by interviewing the patient 106. A web-based user interface 300 running, for example, on a computer 114 of the patient 106 and/or a computer 116 of the physician 112 can be used to input the patient data 104 (see FIG. 3).

The patient data 104 is transmitted to the server 102 over a network 118 (e.g., the Internet). The patient data 104 can be encrypted to maintain its confidentiality. Upon receipt at the server 102, the patient data 104 can be stored in a database 120. Software running on the server 102 automatically analyzes the patient data 104 and determines a recommended change 122 (e.g., in the insulin dosage of the patient 106) as needed. Thus, the software on the server 102 can identify problems based on the patient data 104 and determine the appropriate recommended change 122. The recommended change 122 determined by the software on the server 102 should be substantially the same as the physician 112 would recommend if he or she were manually analyzing the patient data 104.

The recommended change 122 can be transmitted from the server 102 to the patient 106 and/or the physician 112 over the network 118 (e.g., via e-mail or text message). Depending on any potential risks associated with the recommended change 122, the recommended change 122 can be communicated directly to the patient 106 or to an intermediary (e.g., the physician 112) by the system 100. In an exemplary embodiment, the recommended change 122 could be used to automatically adjust the insulin dosage being delivered to the patient 106 by the pump 108.

The software running on the server 102 uses CBR to determine the recommended change 122. In CBR, solutions to problems previously experienced by each patient, such as the patient 106, are stored in a case base or case library 124. Thereafter, when the patient 106 or a similar patient has the same or a similar problem, an appropriate solution can be retrieved from the case library 124. The case library 124 represents the knowledge for the CBR component of the system 100. A full CBR cycle may be viewed as a process of retrieving a useful past case (i.e., a solution that was successful in addressing a previously encountered problem), reusing the retrieved solution, revising the solution in light of the current problem, and retaining the revised solution as a new case for future use.

A case 126 represents knowledge by storing: (a) the description of a specific problem that has occurred; (b) the solution that was applied to that particular problem; and (c) the outcome of applying the solution to that problem. In describing a problem, it is ideal to include all information that is explicitly taken into account by a human problem solver in solving the problem as well as all information that is typically used in describing such a problem. Typical information for describing a problem can be found in the medical records of the patient 106 and via the software used with the pump 108 and/or the glucose meter 110. Such information can include, for example: blood glucose target levels, actual blood glucose levels, insulin sensitivity, carbohydrate ratios, type of insulin used, basal rates of insulin infusion, bolus doses of insulin with food consumption and/or for correction, type of bolus wave, meal times and an amount of carbohydrates consumed at each meal.

The information explicitly taken into account by a human problem solver in solving each problem can be challenging to identify and acquire. In an exemplary embodiment, knowledge engineering techniques, including shadowing and interviewing physicians (e.g., the physician 112), are used in addition to careful case analysis to determine the case features. Information explicitly considered by a human problem solver in solving blood glucose control problems can include, for example: time of change of insulin infusion set (usually every three days); location of insulin infusion set; mechanical problems with the pump; actions taken to self-correct for hypoglycemia; specific foods consumed at each meal; alcohol consumption; time, type, duration and intensity of exercise; sleep cycles; menstrual cycles; stress and illness.

Solutions to problems usually, but not always, involve changes in insulin dosage. Such changes may be to the amount of basal insulin taken at different times of the day, depending on the amount of physical activity during particular time periods, the amount of bolus insulin used for each meal or correction, or the waveform of a bolus to suit particular foods consumed. Solutions may also involve changes in nutrition, exercise, treatment for hypoglycemia, alcohol consumption, the timing of insulin infusion set changes, the site of insulin infusion set placement, or other lifestyle factors.

A proposed solution may: fix a problem; improve, but not entirely resolve, a problem; fix one problem, but cause another; or fail to fix a problem. When a proposed solution fails to fix a problem, the role of patient non-compliance must be considered as a potential cause or contributing factor to the failure. For example, if the patient 106 is advised to increase his or her bolus dosage, the patient 106 might refuse to do so fearing potential hypoglycemia. Increasing the bolus dosage is still an appropriate recommendation, but to achieve compliance by the patient 106, must be followed up with additional education and reassurance. This additional education and reassurance may be viewed as a modification of, or repair to, the original unsuccessful solution. In general, when a solution is unsuccessful, it may be repaired or replaced by an alternate solution.

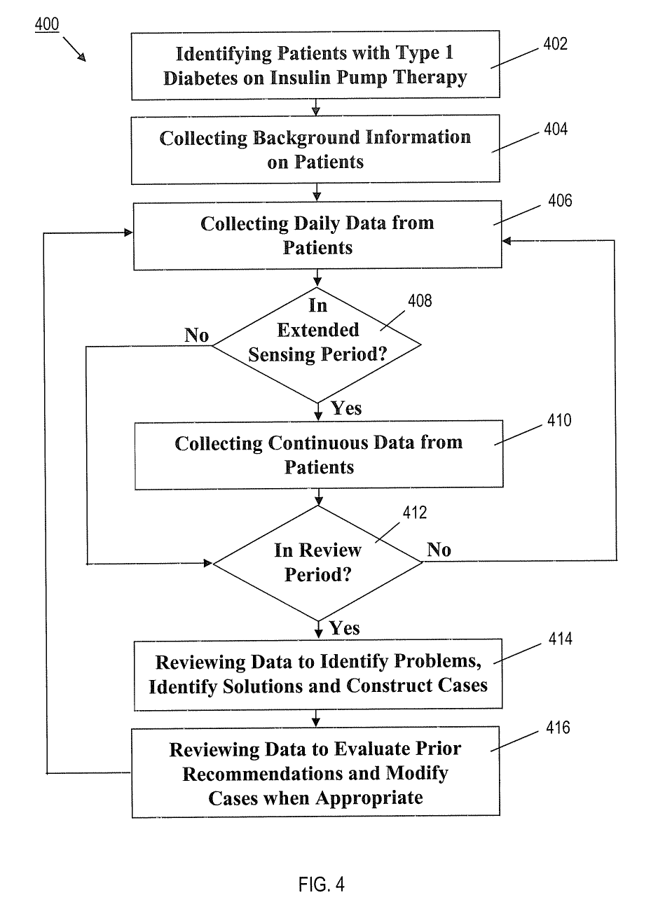

The case library 124 is a data store that holds the cases 126, which represent the knowledge for the system 100. A method 400 for compiling and maintaining the case library 124, according to an exemplary embodiment, is shown in FIG. 4. In general, it is not possible to retroactively construct the cases 126 from existing clinical records. One reason is because much of the data required to construct the cases 126 is not routinely maintained in clinical records. Another reason is that clinical visits are often scheduled months apart (e.g., every three to four months), while blood glucose levels fluctuate continuously. Observing the effects of recommended therapy adjustments (e.g., the recommended change 122) requires more frequent (e.g., daily) updates of the patient data 104. Ideally, the patient data 104 is captured in real time.

In another exemplary embodiment, the functionality of the server 102 (including the software thereon) is transferred to the pump 108 itself, such that the server 102 is no longer needed.

In FIG. 4, the cases 126 in the case library 124 are obtained by identifying a group of patients with type 1 diabetes that are on insulin pump therapy (e.g., the patient 106). See step 402. Background data on the patients is collected in step 404. As noted above, this background data can include, for example, biographical data (e.g., (e.g., time of awakening, meal times)), information on the pump 108, insulin sensitivity, carbohydrate ratios, HbAlc as a measure of long-term blood glucose control, complications from diabetes, other chronic diseases, medications, family history of diabetes and typical daily schedules for work, exercise, meals and sleep. The background data can be part of the patient data 104. The background data is sent to the server 102 over the network 118 where it can be used to construct the cases 126 in the case library 124.

The patients are then monitored for a period of time. The monitoring of the patients can be part of a formal study. In an exemplary embodiment, at least twenty patients are monitored for at least six weeks. Periodically (e.g., daily) during the monitoring period, each of the patients provides his or her actual daily data, according to step 406. The daily data can include, for example, six to ten daily blood glucose readings from finger sticks, bolus dosages and waveforms, basal rates, work schedules, sleep schedules, exercise, meals, infusion set changes, hypoglycemic episodes, menstrual cycles, stress and illness. Furthermore, the patients are encouraged to input information about any miscellaneous events that they believe could be impacting their blood glucose levels. The daily data can be part of the patient data 104. The patients can input their daily data using the web-based user interface (e.g., running on the computer 114 of the patient 106), thereby allowing convenient entry of the daily data at anytime. The daily data is sent to the server 102 over the network 118 where it can be used to construct the cases 126 in the case library 124.

At least a portion of the period of time that the patients are monitored is a period of extended sensing. See step 408. For example, during a six-week monitoring period, days 1-3, 15-17 and 40-42 can be designated for extended sensing. During the extended sensing, the patients wear a device (e.g., the glucose meter 110) that allows for more frequent blood glucose readings (e.g., every five minutes), according to step 410. Thus, the extended sensing provides extended data which greatly expands on the daily data available from the routine daily finger sticks. In an exemplary embodiment, the patients wear the device at least three times with each time lasting at least three days. The extended data can be part of the patient data 104. The extended data can be automatically sent to or retrieved by the server 102 over the network 118 where it can be used to construct the cases 126 in the case library 124. For example, at the end of each extended sensing period, the extended data is downloaded to the database 120.

In an exemplary embodiment, the entire period of time that the patients are monitored is the period of extended sensing.

It is determined in step 412 if the patient data 104 (i.e., the background data, the daily data and/or the extended data) should be reviewed. The patient data 104 is reviewed periodically (e.g., once a week) by physicians to identify new problems and recommend therapy adjustments (e.g., the recommended change 122), according to step 414.

Various tools can be used to help the physicians or other health care providers interpret the large volume of information in the patient data 104. For example, a written report can be used to describe the daily data and the extended data that was collected over the past time interval (e.g., week), as well as the background data of the patients. As another example, a visual representation (e.g., a graph) can be used to assist in analyzing this data.

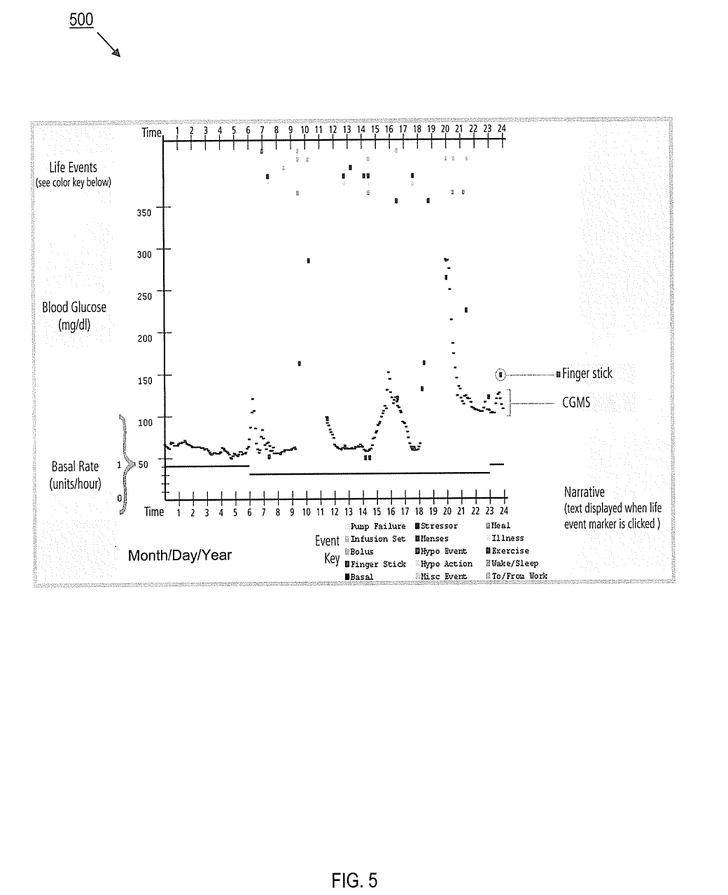

In an exemplary embodiment, as shown in FIG. 5, software running on the server 102 displays life-event data, glucose levels and insulin therapy information for the patient 106 in the form of a graph 500. In the graph 500, the horizontal axis indicates a period of time (e.g., 24 hours for a given date, i.e., month/day/year) over which the data was sampled or the events corresponding to the data occurred. A first vertical axis, near the middle of the graph 500, indicates blood glucose levels of the patient 106. A second vertical axis, below the first vertical axis on the graph 500, uses line segments to indicate the units of basal insulin infused per hour for the patient 106. A series of color-coded or otherwise differentiated markers located near the top of the graph 500, above the first vertical axis, depict other types of clinical data (e.g., time of awakening, meal times) collected on the patient 106, as well as problems or other occurrences (e.g., a hypoglycemic episode, pump failure) related to the patient 106. In an exemplary embodiment, the graph 500 is interactive such that if the physician 112 reviewing the data clicks on one of the markers, additional narrative information is displayed that relates to the event corresponding to the clicked marker. For example, clicking on a marker corresponding to a hypoglycemic episode of the patient 106 results in the display of information on the hypoglycemic episode (e.g., the time of the episode, the symptoms being experienced by the patient 106 during the episode).

During and/or after the review of the patient data 104 by the physicians, the physicians explain their findings to knowledge engineers. The knowledge engineers have the technical skills to structure these findings into the cases 126 in the case library 124, and do so in step 414.

Thereafter, in step 416, the physicians can evaluate the effectiveness of previously-recommended therapy adjustments. The physicians can also contact the patients to discuss any questions or recommended therapy adjustments, as well as invite the patients to provide their own interpretations of observed trends. In this manner, the physicians can determine if any adjustments need to be made to the cases and, if so, instruct the knowledge engineers to modify the cases, in step 416.

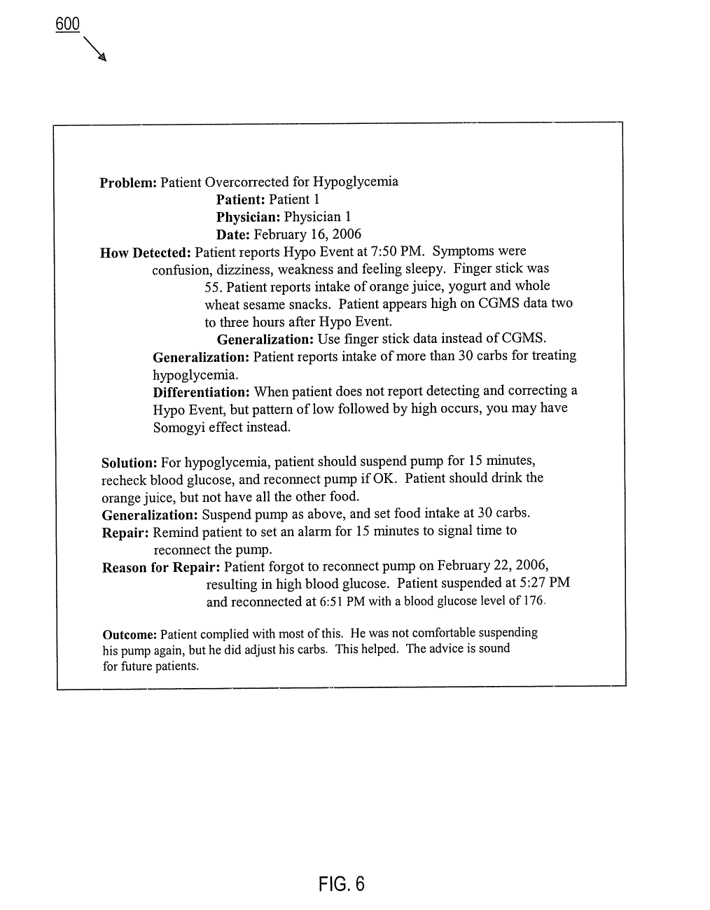

An exemplary case 600 is shown in FIG. 6. By way of background, the patient 106 experienced a hypoglycemic event at 7:50 pm on Feb. 16, 2006, wherein the patient 106 had a blood glucose reading of 55 and experienced symptoms including confusion, dizziness, weakness and fatigue. The patient 106 treated the hypoglycemia by consuming orange juice, yogurt and whole wheat sesame snacks. Shortly after the hypoglycemic episode of the patient 106, both finger stick data and glucose meter data show the patient 106 to be hyperglycemic. Accordingly, the exemplary case 600 identifies the problem as the patient 106 overcorrecting for hypoglycemia.

In describing the self-treatment conducted by the patient 106 in response to the hypoglycemic episode, the patient 106 provided evidence for the likely cause of the ensuing hyperglycemia. In particular, the patient 106 ate and drank more than the recommended 15 to 30 grams of carbohydrates needed to return to a normal blood glucose level. This is an important problem to correct in order to avoid a “roller coaster” pattern of highs and lows. Such a pattern was evident for the patient 106 based on the monitoring.

As shown in the exemplary case 600, the physician 112 recommended a change in the treatment of hypoglycemia for the patient 106. For future hypoglycemic events, the patient 106 was advised to suspend use of the pump 108 for 15 minutes, to take a finger stick reading and to reconnect the pump 108 if the finger stick reading indicated that the blood glucose level was within the target range for the patient 106. The patient 106 was also advised to consume orange juice only, without the yogurt and the whole wheat sesame snacks.

The patient 106 initially complied with the recommendation of the physician 112 (i.e., suspending the pump 108 and drinking only the orange juice). However, the patient 106 forgot to reconnect the pump 108 and thereafter became hyperglycemic. The patient 106 then had to use bolus insulin to correct the hyperglycemia. As a result, the solution for the exemplary case 600 was repaired to advise the patient 106 to set an alarm signaling the time to recheck the blood glucose level and reconnect the pump 108. As the outcome for the exemplary case 600, the patient 106 was no longer willing to risk disconnecting the pump 108 but did adjust carbohydrate intake accordingly. Upon subsequent monitoring, the patient data 104 showed that the patient 106 experienced less hyperglycemia following treatment for hypoglycemia.

The form of the cases 126 (e.g., the exemplary case 600) is a structured or relational representation. Cases in other CBR systems may take varying forms including feature vector representations and textual representations. Compared to these other representations, the relational representation may require more knowledge intensive methods of acquiring, comparing and reusing the cases 126. However, the relevant domain is clearly relational in nature. More important than obtaining absolute information about when the patient 106 awoke, when the patient 106 worked or what the patient 106 ate would be obtaining relative information revealing that the patient 106 awoke later than usual, worked longer than usual or ate something out of the ordinary, like a holiday dinner.

A drawback to using knowledge intensive methods in representing the cases 126 is that additional time and effort can be required on the part of busy domain experts and knowledge engineers. Domains in the health sciences, in particular, are marked by problems that require a lot of knowledge possessed by a few people to solve. Accordingly, there are several ways to minimize such a knowledge engineering bottleneck. For example, specific cases can be used to narrow down the space of all theoretically possible problems to a subset that are known to most commonly occur. As another example, a graphical visualization tool (as described above) can be used to facilitate review of the cases 126. As yet another example, the expertise of clinical nurse practitioners and the patients themselves can be leveraged to parallelize the knowledge engineering process.

The use of patient expertise, although unusual, is warranted in this domain because patients frequently make their own therapy adjustments based on years of experience in managing their own diabetes. For example, if the patient 106 has a child that causes the patient 106 considerable stress, the patient 106 might bolus just before the child was expected to visit in order to prevent the stress-induced rise in blood glucose levels that would otherwise ensue.

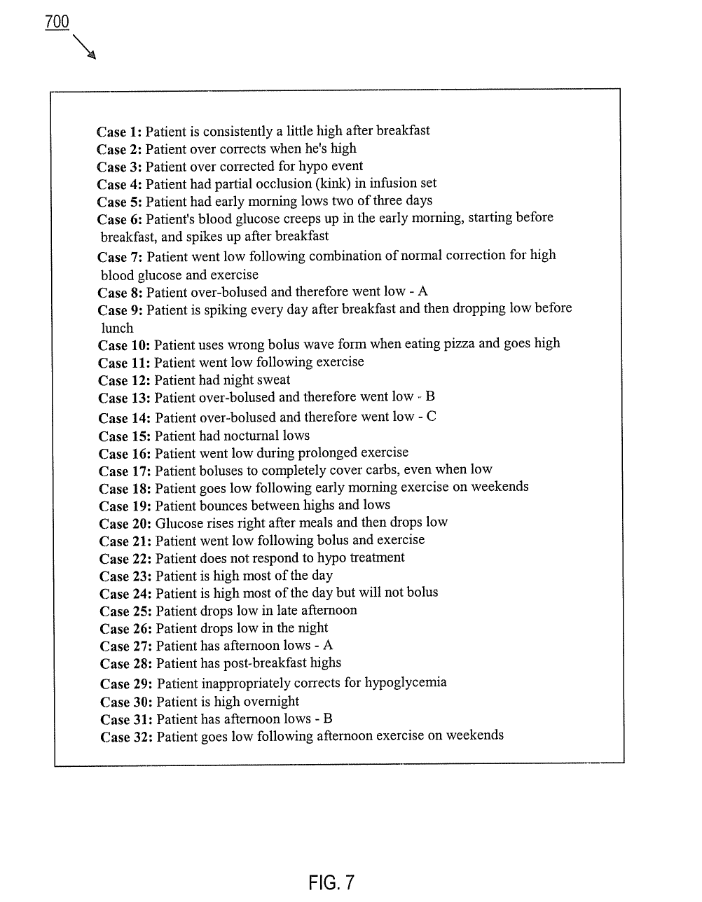

An exemplary case library 700 is shown in FIG. 7. The exemplary case library 700 includes 32 cases 126 that cover a broad range of problems experienced by Type 1 diabetics on insulin pump therapy. One of ordinary skill in the art will appreciate that the case library 700 could include more of fewer cases 126. Furthermore, as the CBR-based system 100 is used by more patients and physicians, the case library 700 will continue to grow as new cases 126 are inserted therein, such that the system 100 will continue to evolve into a more robust system. As noted above, the case library 700 of cases 126 can be stored in the database 120 or some other data store, such that the case library 700 can be readily updated (e.g., in a periodic, on-demand or event-driven fashion) to introduce new cases 126 into the case library 700.

In an exemplary embodiment, all of the cases 126 in the case library 700 were formed based on problems encountered by patients during actual monitoring of the patients. For each problem, a solution was recommended by the physician 112 to the patient 106 experiencing the problem. The outcomes of applying the solutions to the problems were monitored and recorded. As noted above, these problems, solutions and outcomes were used to construct the cases 126.

Using the cases 126 in the case library 124, a current problem being experienced by the patient 106 can be matched to the same or a similar problem, represented in a case 126, that was previously experienced by the patient 106 or a similar patient. For example, the software on the server 102 in the system 100 of FIG. 1 can perform the problem and/or patient matching.

In general, similar problems experienced by the same patient will rank higher than those experienced by different patients, and similar problems experienced by more similar patients will outrank those experienced by less similar patients. This is important for two reasons. First, the same patient often re-experiences problems that he or she has experienced in the past. This is especially likely for problems that occur cyclically over long periods of time, such as only during basketball season or only during pregnancies. Second, in solving problems, physicians take patient characteristics into account to maximize compliance. For example, a solution that works for a middle-aged man might not be acceptable to a teenager contending with peer pressure at school, even though their diabetes-related problems may be very similar.

In an exemplary embodiment, the software running on the server 102 includes routines for comparing a first problem and a second problem to determine a value indicating how similar the first problem is to the second problem. Thus, the software routines form similarity metrics that are useful for matching data representing a current problem (i.e., a newly input case) to an existing case 126 representing a previously encountered similar problem. In this manner, the solution and the outcome associated with the case 126 for the similar problem can be used to provide the patient with the recommended change 122, as needed, for the current problem.

In an exemplary embodiment, one or more functions are called by the software for each comparison. Various exemplary comparison functions for use as the similarity metrics are set forth in Table 1. The functions can involve direct and/or indirect comparison of features between a newly input case and a case (e.g., the case 126) in the case library 124. The result of each function is a score (e.g., 0.00 to 1.00) representing how well the corresponding features of the two cases being compared match each other, with a higher value indicating a closer match.

TABLE 1

| Category | Function | Usage |
|---|---|---|
| Problem Info | compareProblemType | Compares problem type |
| | comparePattern | Compares pattern type |
| | compareSitAsses | Compares situation assessment results |
| Hypo Details | compareRapidDrop | Compares if glucose levels fell rapidly |
| | compareAwareness | Compares if patients were aware of hypo event |
| | compareConsumption | Compares use of carbs to correct hypo event |
| | compareSuspension | Compares suspension of pump to correct hypo |
| | | event |
| Hyper Details | compareRapidRise | Compares if glucose levels rose rapidly |
| | compareExtHigh | Compares if glucose levels were extremely high |
| | compareBolus | Compares use of bolus to correct hyper event |
| | compareInfSetChange | Compares changing of infusion set |
| Other Factors | CompareRelToBolus | Compares relation to bolus administration |
| | compareRelToDOW | Compares relation to day of week |
| | compareRelToTOD | Compares relation to time of day |
| | compareRelToExer | Compares relation to exercise |
| | compareTempBasal | Compares use of temporary basal rate |
| | compareRelToMeal | Compares relation to a meal |
| | compareRelToFood | Compares relation to a particular food |
| | compareStressors | Compares presence of stressful factors |

Not all of the functions are necessarily computed for each case comparison. To increase efficiency, if a function is determined to be of no value to the comparison of the cases, the function is not called. For example, if a problem is not related to hyperglycemia, then the functions for features related to hyperglycemia are not applicable and need not be called. The execution of a similarity determination module, according to an exemplary embodiment, is represented by the pseudo-code set forth in Table 2.

```
TABLE 2
Determine match score of problem types.
  If problem type match scores are below the threshold for further
  comparison, no further comparisons are performed and the case's
  score is computed.
  If problem type match score is above the threshold for further
  comparison, continue comparing case features.
Compare general problem details as follows:
  Determine match score for situation assessment and add it to the
  aggregate score.
  Determine match score for problem pattern and add it to the
  aggregate score.
If problem type does not concern hypoglycemia, add hypoglycemia detail
factor weights to aggregate subtracted score and continue to next step. If
problem type concerns hypoglycemia, compare hypoglycemia details as
follows:
  Determine match score for rapid decrease in glucose level and add it
  to the aggregate score.
  Determine match score for patient awareness of hypoglycemia and
  add it to the aggregate score.
  Determine match score for corrective consumption and add it to
  the aggregate score.
  Determine match score for insulin pump suspension and add it to
  the aggregate score.
If problem type does not concern hyperglycemia, add hyperglycemia detail
factor weights to aggregate subtracted score and continue to next step. If
problem type concerns hyperglycemia, compare hyperglycemia details as
follows:
  Determine match score for rapid increase in glucose level and add it
  to the aggregate score.
  Determine match score for extremely high glucose level and add it
  to the aggregate score.
  Determine match score for corrective insulin administration and add
  it to the aggregate score.
  Determine match score for infusion set change and add it to the
  aggregate score.
Compare details regarding the cases relationship to other various factors
as follows:
  Determine match score for relationship to bolus administration and
  add it to the aggregate score.
  Determine match score for relationship to day of week and add it
  to the aggregate score.
  Determine match score for relationship to time of day and add it
  to the aggregate score.
  Determine match score for relationship to exercise and add it to
  the aggregate score.
  Determine match score for temporary basal rate and add it to the
  aggregate score.
  Determine match score for relationship to meal and add it to the
  aggregate score.
  Determine match score for relationship to specific food and add it
  to the aggregate score.
  Determine match score for relationship to stress factors and add it
  to the aggregate score.
Determine case match score as follows:
  Determine total possible weight by subtracting the aggregate
  subtracted score from the total importance weight of all factors.
  Divide the aggregate score by the total weight.
Assign score to case.
```

As can be seen from the pseudo-code in Table 2, if two cases are close enough to warrant further comparison, the cases are then compared based on four different categories: general problem details, hypoglycemic details, hyperglycemic details and details regarding the case's relationship to other various factors (see also Table 1). These comparisons are based on Boolean and/or enumerated type values for the features in the cases being compared.

For example, the Boolean comparisons include the rapid glucose level drop, corrective consumption and pump suspension comparisons from the hypoglycemic details category; the rapid glucose level rise, extreme high, corrective bolus and infusion set change comparisons from the hyperglycemic details category; and the related to bolus, related to exercise, temporary basal rate, related to specific food and related to stressful factors comparisons the other various factors category. Pseudo-code for the general operation of a Boolean comparison, according to an exemplary embodiment, is set forth in Table 3.

```
TABLE 3
Determine the degree of match as follows:
  If the value of the feature from the existing case and the value of the
  feature from the new case are either both true or both false, the degree
  of match is 1.0.
  If the value of the feature from one case is true while the value of the
  feature from the other case is false, the degree of match is 0.0.
Determine the feature's match score as follows:
  Multiply the feature's degree of match by the importance weight of the
  feature.
```

Enumerated type comparisons include the problem type, pattern and situation assessment comparisons from the general problem details category; and the patient awareness comparison from the hypoglycemic details category. Pseudo-code for the general operation of a Boolean comparison, according to an exemplary embodiment, is set forth in Table 4.

```
TABLE 4
Determine the degree of match as follows:
  Assign a value in the range of 0.0 to 1.0 based on similarity tables
  which contain the degree of match values for all combinations of
  enumerated type values from the existing case and the new case.
Determine the feature's match score as follows:
  Multiply the feature's degree of match by the importance weight of the
  feature.
```

A combination of Boolean and enumerated type values are used for the comparisons of the related to day of week, related to time of day and related to meal comparisons from the other various factors category. For example, these functions base the decision of whether to compare the enumerated type values of a feature on the Boolean values of another feature.

After the newly input case has been compared against each case in the case library 124 and corresponding match scores have been determined based on the comparisons, the system 100 determines which, if any, of the cases in the case library 124 will be returned.

In an exemplary embodiment, the software running on the server 102 includes routines for returning all cases whose score is above a certain threshold. In another exemplary embodiment, the software running on the server 102 includes routines for returning the K cases having the highest scores, where K is a fixed number. The solutions that are recommended (e.g., the recommended change 122) for the current problem are taken, either directly or after some modification, from the returned cases.

In an exemplary embodiment, the software running on the server 102 includes routines for comparing a first patient and a second patient to determine a value indicating how similar the first patient is to the second patient. Thus, the software routines form similarity metrics that are useful for matching data (e.g., the background data) representing the patient 106 to data representing another patient. One of ordinary skill in the art will appreciate that direct comparison of the background data (e.g., age, gender, occupation) is one useful similarity metric for determining how similar the first patient is to the second patient. In determining the recommended change 122 for the patient 106 experiencing a current problem, it is not only useful to identify a case 126 for a previously encountered similar problem, but one which was previously encountered by the same or a similar patient.

The similarity metrics facilitate retrieval of appropriate cases 126. In particular, the similarity metrics are useful for identifying and retrieving the past cases 126 that are most likely to help current patients with their problems. A good metric typically combines the relevant case dimensions with (1) domain dependent measures of how well two cases match along each dimension and (2) weights describing how important it is for cases to match along each dimension.

In an exemplary embodiment, the software running on the server 102 includes routines for adapting a solution to a previously encountered problem, represented in a case 126, to better fit a currently encountered similar problem. Thus, the software routines implement adaptation strategies that enable the modification of solutions found in prior cases 126 to best solve current problems. In this manner, new cases can be constructed by modifying existing cases. Often, the adaptation strategies involve parameter adjustment. For example, a patient who has low blood sugar in the afternoons may adjust his or her afternoon insulin basal rate from 2.0 units per hour to 1.8 units per hour. In applying this adjustment to future patients, one must consider the patient's current basal profile and adjust it accordingly, rather than simply transferring the value 1.8. Other adaptation strategies are more domain specific. For example, if a patient requires additional education and reassurance to insure compliance with one adjustment, that requirement may be added to future adjustments for that patient or for similar patients.

The above description of specific embodiments has been given by way of example. From the disclosure given, those skilled in the art will not only understand the general inventive concept and its attendant advantages, but will also find apparent various changes and modifications to the structures and methods disclosed. For example, although the above exemplary embodiments have been described in the context of patients with type 1 diabetes on insulin pump therapy, the general inventive concept is broader and could be adapted to patients with other types of diabetes (e.g., type 2 diabetes) and/or on other types of medications (e.g., oral medications), as well as to other types of diseases. It is sought, therefore, to cover all such changes and modifications as fall within the spirit and scope of the general inventive concept, as defined herein, and equivalents thereof.

What is claimed is:

1. A method for managing diabetes in a patient with diabetes, the method comprising:
collecting data on the patient;
analyzing the data to determine if a therapeutic adjustment is needed in response to a current situation;
if the therapeutic adjustment is needed, using case-based reasoning to match the current situation to a prior situation to determine the therapeutic adjustment; and
outputting the therapeutic adjustment.

2. The method of claim 1, wherein the data includes a blood glucose level of the patient.

3. The method of claim 1, wherein the data includes at least one of a work schedule of the patient, an exercise schedule of the patient, a meal schedule of the patient and a sleep schedule of the patient.

4. The method of claim 1, wherein the collecting the data includes at least one of receiving information provided by the patient, receiving information provided by a physician of the patient and receiving information from a device attached to the patient.

5. The method of claim 1, wherein the analyzing the patient data includes determining if a blood glucose level of the patient is within a predetermined range of blood glucose levels.

6. The method of claim 1, wherein the current situation is the patient is hypoglycemic.

7. The method of claim 1, wherein the current situation is the patient is hyperglycemic.

8. The method of claim 1, wherein the case-based reasoning is the sole modality for determining the therapeutic adjustment.

9. The method of claim 1, wherein the case-based reasoning is the primary modality for determining the therapeutic adjustment.

10. The method of claim 1, wherein the prior situation was experienced by the patient.

11. The method of claim 1, wherein the prior situation was experienced by another patient.

12. The method of claim 1, wherein the patient has Type 1 diabetes and is on insulin pump therapy.

13. The method of claim 12, wherein the outputting the therapeutic adjustment includes adjusting an insulin dosage delivered by an insulin pump to the patient.

14. The method of claim 1, wherein the collecting the data and the analyzing the data occur periodically at a predetermined interval.

15. The method of claim 14, wherein the predetermined interval is less than or equal to 5 minutes.

16. The method of claim 1, further comprising evaluating the outcome of the therapeutic adjustment.

17. A system for managing diabetes in a patient, the system comprising:
a client; and
a server including software,
wherein data on the patient is transmitted from the client to the server;
wherein the software on the server analyzes the data to determine if a therapeutic adjustment is needed in response to a current situation;
wherein, if the therapeutic adjustment is needed, the software on the server uses case-based reasoning to match the current situation to a prior situation to determine the therapeutic adjustment; and
wherein the software on the server outputs the therapeutic adjustment.

18. The system of claim 17, wherein the client is operable to communicate with the server over a network.

19. The system of claim 18, wherein the network is the Internet.

20. The system of claim 17, further comprising a case library,
wherein the case library stores a plurality of cases and each case includes a problem, a solution and an outcome of applying the solution to the problem.

21. The system of claim 20, wherein, if the therapeutic adjustment is needed, the software on the server uses case-based reasoning to identify at least one case that approximates the current situation.

22. The system of claim 21, wherein the software on the server evaluates the outcome of applying the solution from the at least one case to the current situation.

23. An apparatus for managing diabetes in a patient, the apparatus comprising:
an insulin pump for delivering insulin to the patient; and
a data processing unit,
wherein the data processing unit receives data on the patient and analyzes the data to determine if a therapeutic adjustment is needed in response to a current situation;
wherein, if the therapeutic adjustment is needed, the data processing unit uses case-based reasoning to match the current situation to a prior situation to determine the therapeutic adjustment.
